(19) United States
(12) Patent Application Publication
(10) Pub. No.: US 2022/0379023 A1
(43) Pub. Date: Dec. 1, 2022

(54) ENTERAL FEEDING LIQUID DELIVER

(71) Applicant: KPR U.S., LLC, Mansfield, MA (US)

(72) Inventors: Wayne BIERMANN, St. Charles, MO (US); Laura BRAMES, Chesterfield, MO (US); John HOLSTE, Hamburg, IL (US); Gary WALDHOFF, St. Charles, MO (US); Paul TRELFORD, St. Louis, MO (US)

(21) Appl. No.: 17/750,041

(22) Filed: May 20, 2022

Related U.S. Application Data
(60) Provisional application No. 63/192,462, filed on May 24, 2021.

(51) Int. Cl. A61M 5/168; A61M 5/142

(52) U.S. Cl. CPC A61M 5/16809; A61M 5/14228; A61M 2005/14208

(57) ABSTRACT

An apparatus and method for delivering liquid to a subject using a pumping device of a flow control apparatus. The method includes recognizing a pump set including a liquid container with a volume of liquid mounted to the flow control apparatus whereby the pump set is positioned to be acted on by the pumping device to deliver aliquots of liquid through the pump set. Operation of the pumping device is initiated to draw a prescribed volume of the liquid from the liquid container for a duration of time. A single aliquot of the volume of liquid is delivered from the liquid container to the subject. Operation of the pumping device is paused for a predetermined period of time before delivering another single aliquot of the volume of liquid from the liquid container to the subject.

### CROSS-REFERENCE TO RELATED APPLICATION

This application claims priority to U.S. Provisional Patent Application No. 63/192,462, entitled “ENTERAL FEEDING LIQUID DELIVERY,” filed on May 24, 2021, which is hereby incorporated by reference in its entirety.

### FIELD

The present invention generally relates to delivery of liquid by an enteral feeding pump, and more particularly to the delivery of a relatively thick liquid by an enteral feeding pump.

### BACKGROUND

Administering medicine or nutrition to a patient who cannot intake the medicine or nutrition orally can be affected by utilizing peristaltic flow control systems. Typically, in such systems, liquid is delivered to the patient by a pump set including a resiliently collapsible elastomeric tubing loaded on a flow control apparatus, such as a peristaltic pump, which delivers liquid to the patient at a controlled rate of delivery. The peristaltic pump usually has a housing that includes a rotor operatively engaged with a motor through a gearbox. The rotor drives liquid through the flexible tubing of the pump set by the peristaltic action effected by reversible compression of the tubing created by impingement, e.g., pinching, by one or more rollers on the rotor. Rotation of the rotor progressively compresses the elastomeric tubing that drives the liquid at a controlled rate. The pump set may have a valve mechanism for permitting or preventing liquid flow communication through the pump set. The flow control system may also have a controller that operatively regulates the one or more motors which effectively controls liquid flow.

Peristaltic pumps operate by delivering liquid in small charges called “aliquots”. The rotor engages elastomeric tubing of the pump set, pinching off a portion of the elastomeric tubing and pushing liquid forward of the pinch point, e.g., closer to the patient than to the source of liquid toward the patient. Typically, the volume of liquid to be administered to the patient is controlled in the pump by counting the number of aliquots, each being of substantially the same volume, and stopping when the number reaches an amount corresponding to the total desired volume of liquid to be delivered. Peristaltic pumps are sanitary and generally accurate and therefore very useful in the administration of medication and therapeutic liquids to the patient.

Current enteral feeding pumping methods can be ineffective when delivering thick liquids, such as nutritional liquids that have high viscosity and/or that contain solids which approximate chewed food for enteral feeding purposes (e.g. blended foods).

### SUMMARY

In one aspect, a method of operating a flow control apparatus to deliver nutritional liquid to a subject using a pumping device of the flow control apparatus generally comprises recognizing a pump set including a liquid container with a volume of liquid mounted to the flow control apparatus whereby the pump set is positioned to be acted on by the pumping device to deliver aliquots of liquid through the pump set. Initiating operation of the pumping device to draw a prescribed volume of the liquid from the liquid container for a duration of time. Delivering a single aliquot of the volume of liquid from the liquid container to the subject and pausing operation of the pumping device for a predetermined period of time before delivering another single aliquot of the volume of liquid from the liquid container to the subject. The predetermined period of time for pausing operation is within the duration of time during which the prescribed volume of the liquid is pumped to the subject.

In one aspect, the method further comprises delivering an entire prescribed volume of liquid to the subject in single aliquot increments.

In one aspect, the method further comprises pausing operation of the pumping device for the predetermined period of time after every aliquot delivery of the aliquots making up the entire prescribed volume of liquid.

In one aspect, the method further comprises pausing operation of the pumping device for between about 0.5 and about 5 seconds.

In one aspect, the method further comprises rotating a rotor of the pumping device to engage the pump set for delivering the single aliquot of liquid to the subject.

In one aspect, rotating the rotor to deliver the single aliquot of liquid comprises rotating the rotor for less than a full rotation.

In one aspect, the method further comprises associating a delivery routine stored in memory of the flow control apparatus with the recognized pump set.

In one aspect, the liquid comprises a nutritional liquid.

In one aspect, the nutritional liquid comprises a solution of blended food.

In one aspect, the liquid has a viscosity of at least 50 cP.

In another aspect, a flow control apparatus for use with a pump set to deliver liquid from a liquid container through the pump set to a subject generally comprises a pumping device capable of acting on the pump set to produce a liquid flow in the pump set during a feeding cycle. A controller is in communication with the pumping device for controlling operation of the pumping device during the feeding cycle for producing the flow of the liquid in the pump set. The controller includes a processor and a memory. The controller is configured to execute in the processor a feeding routine to deliver a single aliquot of the liquid from the liquid container to the subject and pause operation of the pumping device for a predetermined period of time before delivering another single aliquot of liquid from the liquid container to the subject.

In one aspect, the pumping device comprises a rotor including a plurality of rollers configured to contact the pump set to produce the single aliquots of liquid.

In one aspect, the single aliquot is less than the volume of liquid that would be delivered by one full rotation of the rotor.

In one aspect, the single aliquot comprises the volume of liquid disposed between adjacent rollers of the rotor.

In one aspect, the controller is programmed to execute the feeding routine to deliver an entire prescribed volume of the liquid from the liquid container within the feeding cycle.

In one aspect, the controller is programmed to execute the feeding routine upon recognizing the pump set is loaded on the flow control apparatus.

In one aspect, the apparatus further comprises a reader operatively connected to the processor and configured to read the pump set to identify the feeding routine for delivering the liquid in the liquid container.

In one aspect, the controller is configured to calculate the pause between delivery of successive aliquots based on an input of the volume of liquid to be delivered to the subject.

In one aspect, the controller is configured to execute in the processor another feeding routine in which the pump device delivers multiple aliquots of liquid to the subject during a continuous operation of the pumping device.

In yet another aspect, a method of operating a flow control apparatus to deliver liquid to a subject using a pumping device of the flow control apparatus generally comprises recognizing a pump set including a liquid container with a volume of liquid mounted to the flow control apparatus whereby the pump set is positioned to be acted on by the pumping device to deliver aliquots of liquid through the pump set. Initiating operation of the pumping device to draw a prescribed volume of the liquid from the liquid container for a duration of time. Delivering a series of aliquots of the volume of liquid from the liquid container to the subject. The series of aliquots is alternated with a series of pauses in operation of the pumping device for a predetermined period of time.

In still another aspect, a method of operating a flow control apparatus to deliver liquid to a subject using a pumping device of the flow control apparatus generally comprises recognizing a pump set including a liquid container with a volume of liquid mounted to the flow control apparatus whereby the pump set is positioned to be acted on by the pumping device to deliver aliquots of liquid through the pump set. Initiating operation of the pumping device to draw a prescribed volume of the liquid from the liquid container for a duration of time. Delivering a series of aliquots of the volume of liquid from the liquid container to the subject. The series of aliquots is alternated with a series of pauses in operation of the pumping device for a predetermined period of time without any additional pauses between aliquots of the volume of liquid in the duration of time while the prescribed volume of liquid is pumped to the subject. The series of aliquots and the series of pauses in operation are at uniform intervals within the duration of time during which the prescribed volume of the liquid is pumped to the subject.

### BRIEF DESCRIPTION OF THE DRAWINGS

FIG. 1 is a fragmentary, perspective view of the feeding system including the enteral feeding pump, and part of the feeding set assembly;

FIG. 2 is the perspective view of FIG. 1, but with portions of the cassette removed;

FIG. 3 is a front perspective view of the enteral feeding pump;

FIG. 4 is a perspective of the cassette;

FIG. 5 is a diagram of an embodiment of a mounting member and identification members of the feeding set and further illustrating a related reader device;

FIG. 6 is a block diagram showing components of the enteral feeding pump that may be utilized to implement one or more aspects disclosed herein;

FIG. 7 is a flow chart of a feeding routine of the feeding system;

FIG. 8A is a graph of a feeding routine of the feeding system; and

FIG. 8B is another graph of a feeding routine of the feeding system.

Corresponding reference characters indicate corresponding parts throughout the drawings.

### DETAILED DESCRIPTION

One or more aspects of the present disclosure pertain to peristaltic pumps such as linear and rotary peristaltic pumps and particularly to a rotary peristaltic pump for providing a liquid delivery apparatus that accurately detects and controls the amount of liquid delivered to a patient and dynamically adapts liquid delivery to the patient based on the type of liquid being delivered. Any one or more advantageous features or structures that provide or facilitate any one or more of such features may be implemented in a peristaltic pump employed in various commercial and industrial applications. Thus, although the detailed discussion is directed to an enteral feeding pump with a feeding set assembly including a cassette, any one or more features of the disclosure may be embodied or implemented in other peristaltic pumps. For example, although the exemplarily discussed pump is a rotary peristaltic enteral feeding pump, the present disclosure has application to other types of peristaltic pumps (not shown). Additionally, one or more of the various features and aspects of the disclosure may be implemented in peristaltic pumps that use mechanisms other than rollers without departing from the scope of the present disclosure such as linear peristaltic pumps. Moreover, feeding set assemblies (not shown) that do not include cassettes may also be used within the scope of the present disclosure.

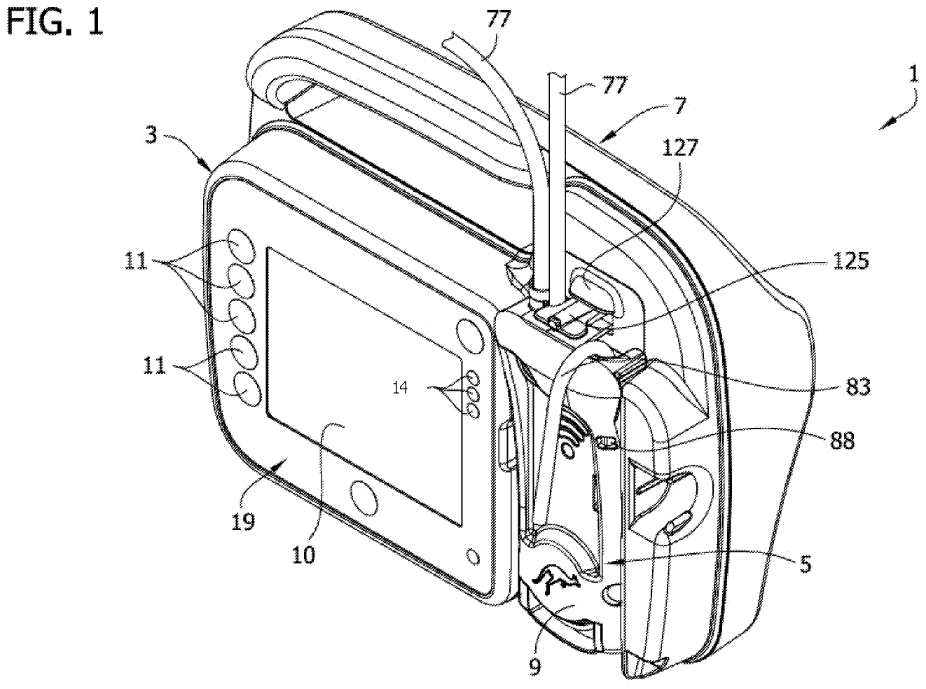

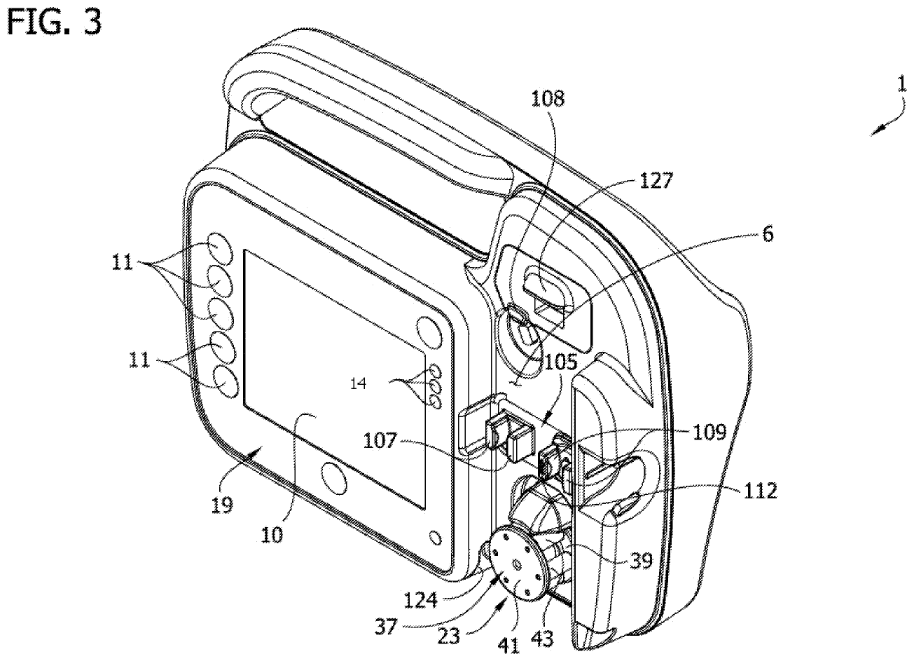

Referring now to the drawings, and in particular FIGS. 1-3, an exemplary enteral feeding pump (broadly, “flow control apparatus”) constructed according to any one or more of the principles of the present disclosure is generally indicated at 1. The feeding pump may comprise a housing generally indicated at 3 that is constructed so as to mount a cassette, generally indicated at 5, of a feeding set assembly (broadly, a “pump set”), generally indicated at 7. The feeding set assembly 7 may include one or more liquid containers (not shown) connected to the cassette 5 via tubing 77. The cassette 5 of the feeding set assembly 7 is releasably attachable to the housing 3. In the illustrated embodiment, a cassette shell 9 of the cassette is removably received in a cassette recess 6 (FIG. 3) in the housing 3. It will be appreciated that “housing” as used herein may include many forms of supporting structures (not shown), including without limitation multi-part structures and structures that do not enclose or house the working components of the pump 1. The pump 1 may also have a display screen 10 on the housing 3 capable of displaying information about the status and operation of the pump. Moreover, various aspects and features of the present disclosure can be implemented without the recess 6. One or more buttons 11 which can be proximate the display screen 10 can be provided for use in controlling and obtaining information from the pump 1, and one or more suitable indicators, such as light emitting diodes 14 can provide status information for the pump.

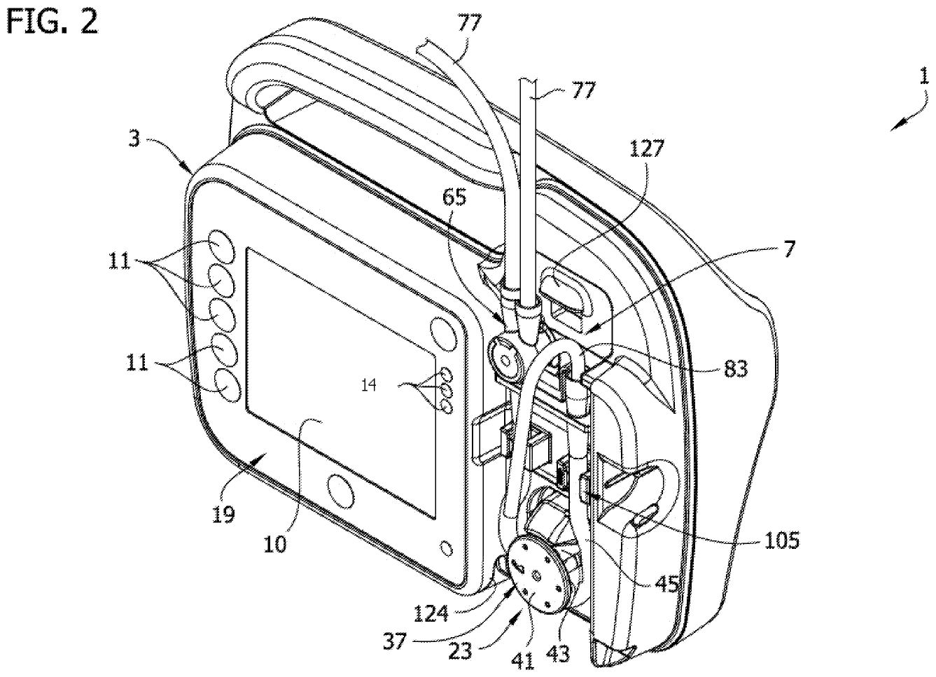

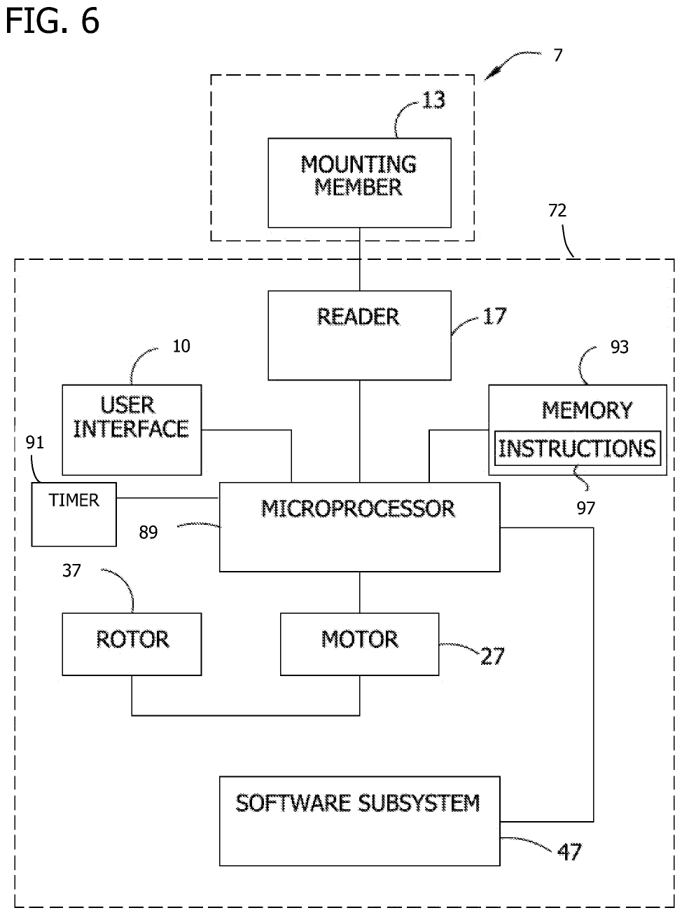

The display screen 10 may be part of a front panel (generally indicated at 19) of the housing 3 and may be removably attached to the housing. The enteral feeding pump may further include a pumping unit indicated generally at 23 (FIGS. 2 and 3) comprising a pump motor 27 (FIG. 6) connected to a rotor shaft (not shown). A battery (not shown) may be received in the housing 3 for powering the pump motor. A power source other than or in addition to the battery could be used to energize the pump including one or more prime motors which drive the pumping unit through the rotor shaft.

The pumping unit 23 has a rotor (generally indicated at 37) which can be coupled to the rotor shaft. The rotor 37 may include an inner disk 39, an outer disk 41, and six rollers 43 (only three of which may be seen in the drawings) mounted between the inner and outer disks for rotation relative to the disks about their longitudinal axes. The rollers 43 engage a tube 45 (FIG. 2) of the feeding set assembly 7 that forms part of the cassette 5 to deliver liquid through the feeding set assembly 7 to a subject when the cassette 5 is attached to the housing 3. For example, nutritional liquid (e.g., blended fruits, vegetables, etc.) may be delivered to a patient using the pump 1, cassette 5, and feeding set assembly 7. Other liquids may also be delivered using the pump 1 without departing from the scope of the disclosure. In the illustrated embodiments, the liquid in the liquid containers is drawn from the containers by the pumping unit 23.

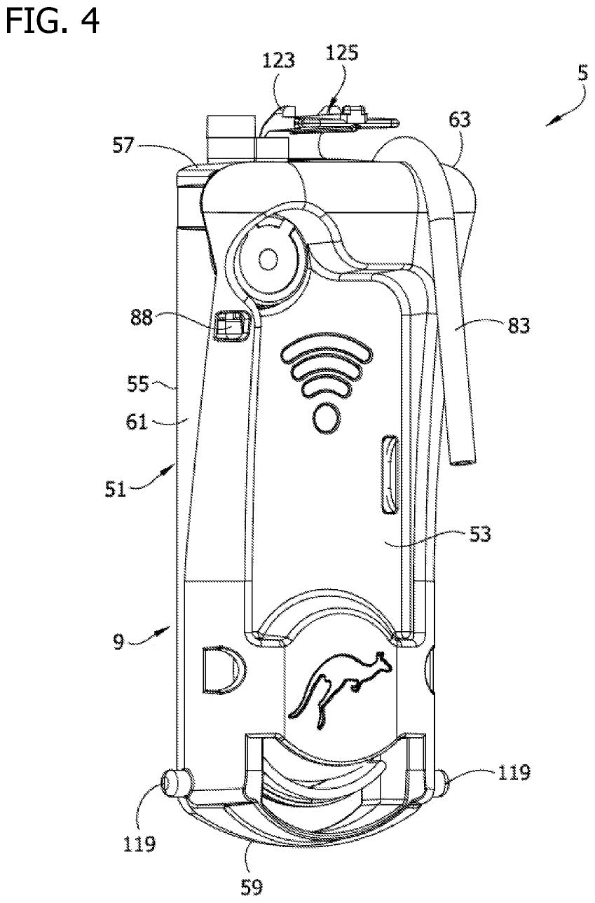

Referring to FIG. 4, the cassette shell 9 comprises a cassette body 51 having a front 53, a back 55, a top 57, and a bottom 59. Side walls 61 and top wall 63 may extend from the back 55 of the cassette body 51 forming a back cavity configured for receiving a fitting 65 (FIG. 2). The tube 45 may be releasably attached to the fitting 65. The fitting 65 may have tabs (not shown) that allow the fitting 65 to be secured or snapped into the cassette. In some cases, the fitting can be removably secured to the cassette. In a one embodiment, the cassette shell 9 mounts the tubing 45 and the fitting 65, and can be made from a polymeric material such as polycarbonate.

Referring to FIGS. 1 and 2, the inlet tubing 77, tube 45, fitting 65, and outlet tubing 83 are considered part of the feeding set assembly 7. The cassette 5 is considered to be part of the feeding set assembly 7 for purposes of this description. The liquid containers may also be considered part of the feeding set assembly 7. However, feeding set assemblies including more of fewer components than described herein are within the scope of the present invention.

Referring to FIGS. 2 and 3, an insert 105 may be received in the cassette recess 6 in the housing 3 to aid in securing the cassette shell 9 and tube 45 in the cassette recess. The insert 105 may be positioned in the recess 6 such that the insert 105 is received in the back cavity of the cassette shell 9 when the cassette is attached to the housing 3. The insert 105 may comprise a pair of opposing first projections 107 disposed at an inlet side of the insert for receiving the inlet portion of the tube 45, and a pair of opposing second projections 109 disposed at an outlet side of the insert for receiving the outlet portion of the tube. Indicia 112 may be disposed on at least one of the second projections 109 indicating the direction of liquid flow in the tube 45. In the illustrated embodiment, the indicia 112 is in the form of an arrow.

To attach the cassette 5 to the pump housing 3, one or more pins or raised projections 119 (FIG. 4) at the bottom 59 of the cassette body 51 of the cassette shell 9 may be inserted in slots 124 (FIGS. 2 and 3) at the bottom of the recess 6 in the housing 3. The engagement between the raised projections 119 and slots 124 generally locates the cassette shell 9 on the housing 3. The cassette body 51 can then be rotated up until ledges 123 on a resiliently deflectable tab 125 at the top 57 of the cassette body are captured by a catch 127 at the top of the recess 6 (FIGS. 2 and 3) to temporarily secure the cassette 5 to the pump 1. To detach the cassette 5 from the pump housing 3, the tab 125 can be depressed to disengage the ledges 123 from the catch 127. Once the cassette 5 is attached to the pump housing 3, the tube 45 extends around a lower portion of the rotor 37 and is positioned for sequential engagement by the rollers 43 of the rotor.

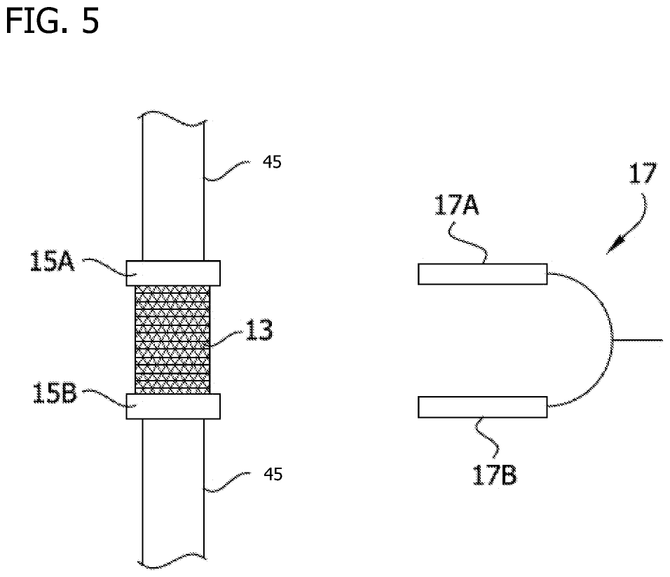

Referring to FIGS. 5 and 6, the feeding set 7 may comprise a mounting member 13 in direct communication with the tubing 45 and one or more identification members 15 on the mounting member. At least one identification member 15 may permit identification of a feeding routine for a nutritional liquid or type of nutritional liquid associated with the feeding set upon engagement of the mounting member 13 to the pump 1. The mounting member 13 may also assist in the loading of the feeding set 7 on the pump 1. However, the mounting member 13 may be omitted and the identification member(s) 15 may be used to load the feeding set 7 to the pump 1. The pump 1 may further include a reader 17 that detects engagement of at least one of the mounting member 13 and the identification member(s) 15 with the pump. In one embodiment, the feeding set 7 may be configured for delivering a relatively thick nutritional liquid. In this instance, the identification member(s) 15 may be configured and/or arranged to indicate a feeding routine for a nutritional liquid in the feeding set 7 that is relatively thick. Additionally, the tubing 77, 45, 83 of the feeding set 7 may also be configured for delivering a thick nutritional liquid. For example, a diameter of the tubing 77, 45, 83 may be increased to provide sufficient cross-sectional area for delivering the thicker nutritional liquid. Still other configurations of the tubing are envisioned without departing from the scope of the disclosure. In one embodiment, a thick nutritional liquid is characterized as a nutritional liquid having a viscosity of at least 50 cP. In another embodiment, a thick nutritional liquid is characterized as a nutritional liquid having a high viscosity and/or containing solids which approximate chewed food for enteral feeding purposes. For example, this can be blended foods including solid food that has been minced in a blender with water or other liquid added to create a multi-state liquid which includes solid, suspension, and liquid portions. Blended foods may also comprise an inconsistent mixture of solid food and liquid that can produce discontinuities in density and viscosity within the mixture when pumped through a feeding set. Additionally, the International Dysphagia Diet Standardisation Initiative (IDDSI) has developed a standardized way of naming and describing texture modified foods and thickened liquids. In one embodiment, a thick nutritional liquid comprises a liquid that registers between a 2 and 4 on the IDDSI Framework. Reference is made to the website www.IDDSI.org, the entire contents of which are hereby incorporated by reference.

An effective flow rate for the pump 1 can depend on a resistance of the tubing of the feeding set 7 and the liquid being delivered through the feeding set. Depending on the desired feeding routine, feeding sets of different constructions can be used with the pump 1. The pump 1 can be configured to recognize automatically the type of feeding set installed and a nutritional liquid associated with the feeding set, and alter or dynamically adapt operation of the pump to accommodate the feeding set and nutritional liquid. In particular, a feeding routine for delivering liquid associated with the loaded feeding set 7 can be automatically customized by retrieving identification information or data represented by the identification member(s) 15 indicating at least one of the type of feeding set, the associated nutritional liquid, and/or characteristics of the nutritional liquid pertaining to delivering the liquid through the feeding set. Such technical features can advantageously effect delivery of the nutritional liquid to the patient by reducing the likelihood against an inappropriate or erroneous delivery protocol. For example, a feeding set having an identification member can provide a representation of the nutritional liquid in the container connected to the pump which in turn can automatically deliver the nutritional liquid according to a predetermined protocol or schedule, which reduces the likelihood of erroneously delivering the nutritional liquid at a different delivery protocol or schedule.

The mounting member 13 is configured to engage opposing second projections 109 (broadly, a mount) of the pump 1 when loading the feeding set 5 on the pump such that the reader 17 may detect the presence of the identification member 15 attached to the mounting member 13. The reader 17 may be disposed on, in, or near the second projections 109 to detect the presence of the identification member 15. In the illustrated embodiment, identification member 15 comprises a first identification component 15A and a second identification component 15B. Any number of identification components is envisioned. The reader 17 may comprise a pair of reader devices 17A, 17B that detect the identification components 15A, 15B, respectively. It will be understood that the number of reader devices 17 may be the same as the number of identification components 15 or different in number. The identification components 15A, 15B may be magnetic components or, in the alternative, magnetically susceptible metallic components capable of detection by reader devices 17A, 17B, respectively without requiring direct physical contact with the reader. The reader devices 17A, 17B may preferably be Hall-effect sensors or other types of proximity sensors that are positioned near the second projections 109 such that the reader devices 17A, 17B can detect the presence of the identification components 15A, 15B when the mounting member 13 is engaged to the mount. Other types of readers may be used. For example, the readers may rely on optically identifying any of the one or more identification components. The identification member 15 can be mounted directly on the cassette 9 and the reader 17 can be positioned to detect the presence of the identification member on the cassette when the cassette is received in the recess 6 of the pump 1.

Upon engagement of the mounting member 13 to the second projections 109, reader devices 17A, 17B may be capable of identifying identification data represented by the number and position of the identification components 15A, 15B. In particular, the attachment of one or more identification components 15A, 15B to the mounting member 13 provides a means for allowing software subsystem 47 (FIG. 6) to identify information related to a nutritional liquid associated with the feeding set 7 loaded on the pump 1. Referring to FIG. 5, the mounting member 13 may have one or more identification components 15A, 15B (two are shown in FIG. 5) attached thereto in accordance with an identification scheme that permits software subsystem 47 to identify the feeding routine for the nutritional liquid associated with the feeding set 7 loaded on the pump 1. In order to identify the feeding routine, a processor such as microprocessor 89 may be operatively associated with memory 93 containing one or more identification schemes for identifying the feeding routine.

With the liquid container(s) attached to the tubing 77, the pump 1 is configured for delivering the feeding solution in the container(s) to a subject. Operation of the pump 1 causes the rollers 43 to engage the tube 45 in the cassette shell 9 to pump the feeding solution from the container(s) to the subject. Engagement of the tube 45 by a roller 43 causes the rollers 43 to initially collapse and occlude the tube 45. Thus, as the rotor 37 rotates to occlude the tube 45 with the rollers 34, liquid is first drawn out of the container (s) through the inlet tubing 77 to be pumped by the pump 1 into the outlet tubing 83 to the subject.

The pump 1 can be programmed or otherwise controlled for operation in a desired manner. For instance, the pump 1 can begin operation to provide feeding to the subject. A user such as a caregiver, or the subject themselves, may select (for example) the amount of liquid to be delivered, the flow rate of the liquid, and the frequency of liquid delivery. The pump 1 may have a controller 72 (FIG. 6) including a processor such as the microprocessor 89 that allows it to accept programming and/or to include pre-programmed operational routines, e.g., algorithm, that can be initiated by the user. The controller 72 may also be connected to the pump motor 27 for controlling its operation to actuate the rotor 37.

The amount of feeding liquid that is delivered to the subject is typically controlled by the number of rotations of the rotor 37 (in a counterclockwise direction as viewed in FIG. 2). In one embodiment, the rotor 37 may include six rollers 43 so that each one-sixth of a rotation delivers one aliquot of liquid to the subject. Aliquot refers to a volume of liquid disposed in the tube 45 between consecutive (leading and trailing) rollers 43. It is envisioned that volumes of liquid defined in other ways could be understood to be an “aliquot.” However as used herein, “aliquot” always refers to a volume of liquid that is less than a volume of liquid that could be delivered by one full rotation of the rotor 37. As each roller 43 first engages the tubing 45, it pinches off the tubing thereby closing off an amount of liquid forward (i.e., toward the subject) from the liquid coming from the feeding source. The roller 43 continues in the counterclockwise rotation which pushes the pinched-off volume of liquid forward of the roller, e.g., the aliquot, toward the subject. Finally, the leading roller 43 releases engagement with the tubing 45 at about the same time the trailing roller engages the tubing for pinching it off for delivering the next aliquot of liquid. Thus, when the microprocessor 89 receives a command to deliver a selected liquid flow rate, it would typically calculate the number of rotations within a given period of time that will deliver a number of aliquots producing the desired flow rate. The selected flow rate may be a rate that is input or selected by the doctor, nurse or other caregiver, or may be a default feeding rate pre-programmed into the pump 1. In one embodiment, a single aliquot is delivered by a single rotor movement.

Conventional pumps operate by delivering fluid in a continuous or sporadic manner throughout the duration of a feeding cycle to deliver an entire prescribed volume of the fluid. For example, conventional pumps may deliver fluid at a constant rate where the rotor is rotated at a constant speed to deliver an entire prescribed volume of fluid to the subject. In this instance, the rotor is continuously rotated through the entire feeding cycle, or a portion of the feeding cycle, to deliver the entirety of the prescribed volume of fluid. Conventional pumps may also deliver fluid in an intermittent fashion where a bolus of fluid (i.e., a large portion of fluid) is delivered to the subject in separate feeding segments spaced apart throughout the day or a portion of the day. In this instance, the pump is operated to rotate the rotor through full rotations to deliver a volume of fluid. The pump will then be stopped and subsequently operated again after a predetermined period of time to deliver another volume of fluid to the subject. In all instances of continuous and intermittent/bolus fluid delivery, the rotor is operated through full rotations without any pausing to deliver the fluid to the subject.

However, when delivering thicker feeding liquids (e.g., blended foods), the continuous/full rotation of the rotor 37 produced by conventional pumping methods may cause an unwanted pressure buildup within the tubing 45, 83 due to the increased viscosity of those liquids causing the tubing to collapse and preventing the desired amount of liquid from being delivered to the subject. Accordingly, the controller 72 may include a timer 91 and a memory area 93 storing a set of instructions 97 for determining a liquid specific feeding routine (e.g., flow rate) for the pump 1 based on the nutritional liquid in the liquid container attached to the tubing. For example, the microprocessor 89 of the pump 1 may identify the feeding routine for the nutritional liquid based on the detected identification members 15 of the pump set. In order to control the feeding routine of the pump 1, the microprocessor 89 retrieves from the memory area 93 the set of instructions 97 for implementing the information data represented by the identification members 15. The microprocessor 89 may then apply the data to the set of instruction in the memory area 93 to determine a feeding routine of the pump 1. The microprocessor 89 may then adjust the motor output to produce the feeding routine to achieve a target-feeding rate. The instructions 97 are machine readable instructions on any suitable medium, broadly identified as the memory area 93. These instructions can be executed by the microprocessor 89. The timer 91 may be initiated in a suitable manner when a feeding cycle (broadly, “operation cycle”) is initiated or performed for delivering feeding liquid to the subject. The controller 72 may use this information along with additional parameters of the feeding cycle to compensate for the thick feeding liquid that is being delivered during the feeding cycle.

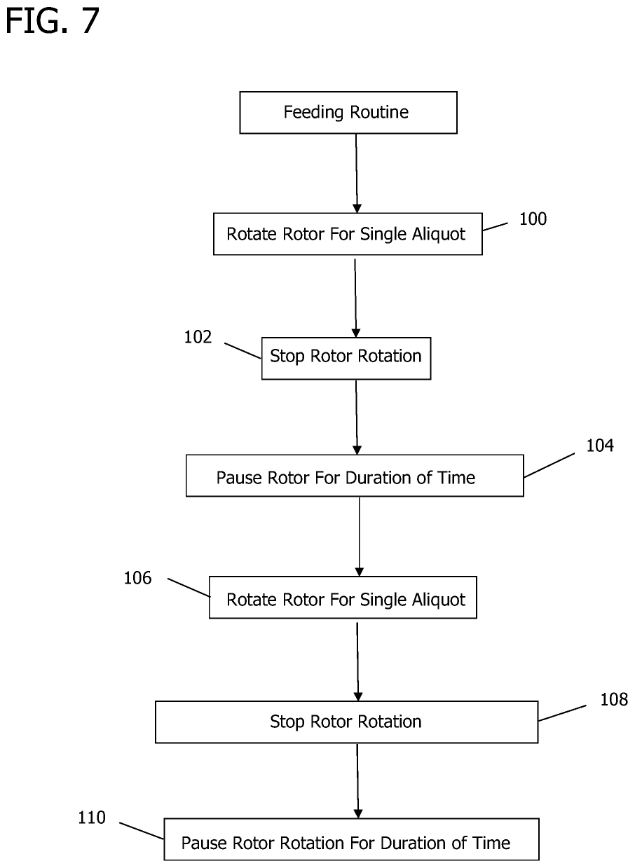

Referring to FIG. 7, the controller 72 can operate to adjust the rotation of the rotor 37 to ensure that the thicker nutritional liquid is properly delivered through the pump set 7. For example, one feeding routine stored in the memory 93 may instruct the controller 72 at 100 to operate the motor 27 to rotate the rotor 37 during a feeding cycle (e.g., 1 hour) to deliver one single aliquot (e.g., ⅓, ¼ or ⅙ of a full turn). The controller 72 may then at 102 instruct the motor 27 to stop rotation of the rotor 37. The rotor rotation may be paused at 104 for a sufficient amount of time to allow liquid from the inlet tube 77 to fill the segment of the tube 45 behind the roller 43, as the roller separates from the tube and the tube rebounds toward its uncollapsed, open state. Failure to provide frequent occurrences of pausing for thicker liquids to fill the rebounding segment of the tube 45 can cause subsequent aliquots to have less than an expected volume, resulting in feeding inaccuracy. In extreme cases, the downstream portion of the tube 45 is evacuated by peristaltic action of the rollers and collapses under the vacuum. The collapsed tube 45 then prevents delivery of further liquid to the patient. In addition, frequent pausing allows time for the aliquot of liquid being delivered to move through at least a portion of the tubing 45, 83 and to allow for any pressure buildup in the tubing to dissipate. The length of time the rotation of the rotor 37 is paused is based on the programmed feeding rate. For a given pump, the amount of time needed to deliver a single aliquot of liquid will be known and stored in the memory 93. Therefore, the total duration of pauses, and therefore the total number of pauses, can be calculated for a given feeding time. Thus, the predetermined period of time for pausing operation occurs within the duration of time during which the prescribed volume of the liquid is pumped to the subject. In general, the greater the amount of liquid to be delivered within the allowed time of a feeding cycle, the shorter amount of time the rotor 37 is paused between aliquots. In one embodiment, the controller 72 operates the motor 27 to pause rotation of the rotor 37 for between about 0.5 and about 5 seconds after a single, small volume aliquot delivery. At 106 the controller 72 then reenergized the motor 27 to rotate the rotor 37 for delivery of another single aliquot, and again stops the rotor at 108 and pauses rotation of the rotor at 110 for the predetermined period of time. This routine is repeated for the entire feeding cycle so that the entire intended volume of liquid is delivered to the subject. The entire intended volume comprises the entire prescribed volume of liquid during a given feeding cycle. In this case, the entire prescribed volume will comprise multiple individual aliquots of liquid. Therefore, the feeding cycle may include the delivery of a series of aliquots of the volume of liquid from the liquid container to the subject alternated with a series of pauses in operation of the pumping device for a predetermined period of time without any additional pauses between aliquots of the volume of liquid in the duration of time while the prescribed volume of liquid is pumped to the subject. In one embodiment, the series of aliquots and the series of pauses in operation are at uniform intervals within the duration of time during which the prescribed volume of the liquid is pumped to the subject.

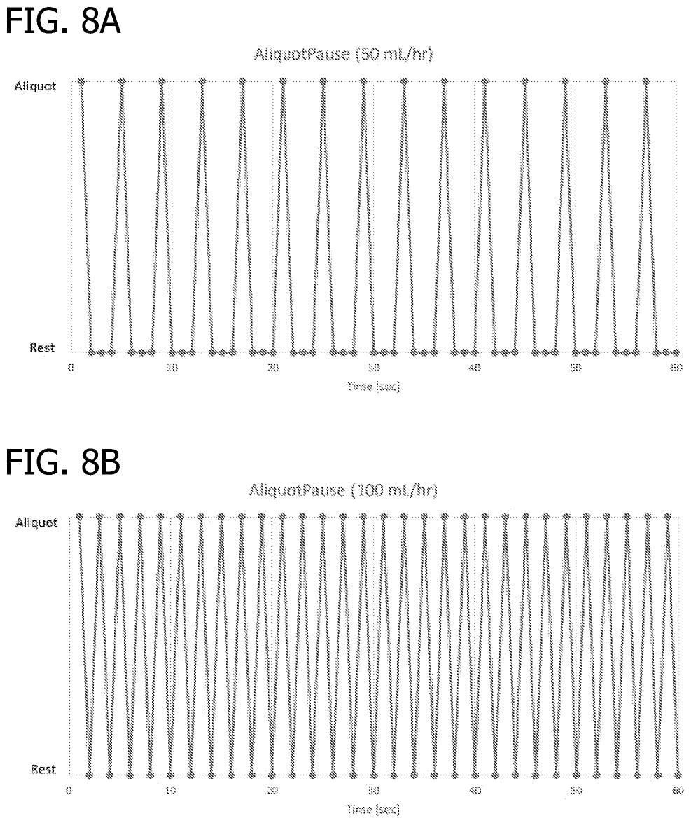

In one embodiment, at least about 90% of the total prescribed volume of thick liquid is delivered to the subject, as compared with only about 70% of the prescribed total volume when the same liquid is delivered using a conventional method. More specifically in the conventional method test, a pump operated a rotor continuously, delivering multiple aliquots to the patient until the prescribed volume for a given period of time (e.g., one minute) was delivered. The pumping was done without regard to the feeding rate. FIG. 8A illustrates a feeding routine according to the present invention for delivering 50 mL of thick nutritional liquid over a 1 hour feeding cycle. FIG. 8B illustrates a feeding routine according to the present invention for delivering 100 mL of thick nutritional liquid over a 1 hour feeding cycle.

Thus, it may be seen that the various objects and features are achieved by the various embodiments disclosed herein. The pump controller 72 allows the microprocessor 89 to adjust the pumping routine for operating the rotor 37 to deliver the feeding liquid through the feeding set 7 to account for the liquid that is being delivered. Therefore, the subject can receive more accurate volume amounts of feeding liquid for a given feeding cycle, particularly with respect to thicker feeding liquids.

In a study conducted comparing existing commercial enteral feeding pumps to enteral feeding pumps incorporating software configured to perform the “aliquot pause” feeding routine as describe above, it was found that enteral feeding pumps with the “aliquot pause” feature exhibited superior performance in fluid delivery when administering “thick” enteral feeding fluid. In particular, the fluid delivery accuracy of the pumps incorporating the “aliquot pause” feature was over 40% more accurate than the existing commercial pumps that did not incorporate an analogous aliquot pause feature.

As represented below in Table 1, an initial study was done to evaluate enteral feeding pump performance of existing commercial pumps based on a standardized testing method. The test method included first classifying the enteral feeding liquid based on the IDDSI Framework. One of the tested enteral feeding fluids included a Real Foods Blend formula of Orange Chicken, Carrots, and Brown Rice. Under the IDDSI Framework the Real Foods Blend measured as a 3 on the IDDSI Framework classifying the formula as a “moderately thick” formula.

Using primed feeding sets, the existing commercial feeding pumps were set at a feeding rate of 25 ml/hr. The pumps were then run for a minimum of 30 minutes. The fluid delivery was when stopped and the feeding rate was adjusted to 125 ml/hr for another 30 minutes. This process was repeated 3-5 times. During each 125 ml/hr cycle, the expected volume of fluid to be delivered was 62.5 ml. The accuracy of fluid actually delivery was calculated as:

$$Accuracy ( percentage ) = \frac{Volume delivered ( ml ) - Expected Volume ( ml )}{Expected Volume ( ml )} \times 100.$$

As can be seen below in Table 1, the existing commercial pumps were incapable of accurately delivering the moderately thick formula. Existing Commercial Pumps 1 and 2 delivered just over half of the expected fluid volume while existing Commercial Pump 3 was not able to deliver any of the moderately thick formula.

TABLE 1
Existing Commercial Pump Fluid Delivery Accuracy

| | | Volume | |
|---|---|---|---|
| Formula | Pump | Delivered (ml) | % Error |
| Real Foods Orange | Commercial Pump 1 | 40 | −36% |
| Chicken, Carrots, | Commercial Pump 2 | 32 | −48% |
| Brown Rice | Commercial Pump 3 | 0 | −100% |

During the “aliquot pause” pump fluid delivery study, two Real Food Blend formulas were compared to water to determine any deviation in the pump's ability to accurately deliver the expected volume of fluid. The fluid delivery cycle was the same as for the test run on the existing commercial pumps. Both food blend formulas registered as “thick” fluids on the IDDSI Framework with the Turkey Blend measuring at a 4 (extremely thick) on the scale, and the Chicken Blend (same as for the study of existing commercial pumps) measuring as a 3 (moderately thick) on the IDDSI scale. As can be seen below in Table 2, there was very little deviation in the pump's ability to deliver the thick fluid formulas as comparted to water. Thus, nearly, 100% of the expected “thick” fluid was delivered using the “aliquot pause” pump. Accordingly, the “aliquot pause” pump performed significantly better than the existing commercial pumps at accurately delivering thick fluids such as the Real Food Blends used in the study.

“Aliquot Pause” Pump Fluid Delivery Accuracy

| | | Volume | |
|---|---|---|---|
| | | Delivered | % Deviation |
| Formula | Pump | (ml) | from water |
| Water | “Aliquot Pause” | 52.93 | N/A |
| | Pump | | |
| Real Food Blends | “Aliquot Pause” | 52.8 | −0.23% |
| Turkey, Sweet Potatoes, | Pump | | |
| and Peaches | | | |
| Real Food Blends | “Aliquot Pause” | 51.62 | −2.47% |
| Chicken, Carrots, and | Pump | | |
| Brown Rice | | | |

Embodiments may be described in the general context of computer-executable instructions, such as program modules, executed by one or more computers or other devices. The computer-executable instructions may be organized into one or more computer-executable components or modules including, but not limited to, routines, programs, objects, components, and data structures that perform particular tasks or implement particular abstract data types. Aspects may be implemented with any number and organization of such components or modules. For example, various features or aspects are not limited to the specific computer-executable instructions or the specific components or modules illustrated in the figures and described herein. Other embodiments may include different computer-executable instructions or components having more or less functionality than illustrated and described herein.

Further, the order of execution or performance of the operations in any of the embodiments illustrated and described herein is not essential, unless otherwise specified. That is, the operations may be performed in any order, unless otherwise specified, and embodiments may include additional or fewer operations than those disclosed herein. For example, it is contemplated that executing or performing a particular operation before, contemporaneously with, or after another operation is within the scope of one or more aspects.

In operation, microprocessor 89 of the controller 72 executes computer-executable instructions such as those illustrated in the figures to implement one or more aspects disclosed herein. Any of the various aspects may also be practiced in distributed computing environments where tasks are performed by remote processing devices that are linked through a communications network. In a distributed computing environment, program modules may be located in both local and remote computer storage media including memory storage devices.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description and shown in the accompanying drawings shall be interpreted as illustrative and not in a limiting sense.

What is claimed:

1. A method of operating a flow control apparatus to deliver liquid to a subject using a pumping device of the flow control apparatus, the method comprising:
recognizing a pump set including a liquid container with a volume of liquid mounted to the flow control apparatus whereby the pump set is positioned to be acted on by the pumping device to deliver aliquots of liquid through the pump set;
initiating operation of the pumping device to draw a prescribed volume of the liquid from the liquid container for a duration of time; and
delivering a single aliquot of the volume of liquid from the liquid container to the subject and pausing operation of the pumping device for a predetermined period of time before delivering another single aliquot of the volume of liquid from the liquid container to the subject, wherein the predetermined period of time for pausing operation is within the duration of time during which an entirety of the prescribed volume of the liquid is pumped to the subject.

2. The method of claim 1, further comprising delivering an entire prescribed volume of liquid to the subject in single aliquot increments.

3. The method of claim 2, further comprising pausing operation of the pumping device for the predetermined period of time after every aliquot delivery of the aliquots making up the entire prescribed volume of liquid.

4. The method of claim 3, further comprising pausing operation of the pumping device for between about 0.5 and about 5 seconds.

5. The method of claim 1, further comprising rotating a rotor of the pumping device to engage the pump set for delivering the single aliquot of liquid to the subject.

6. The method of claim 5, wherein rotating the rotor to deliver the single aliquot of liquid comprises rotating the rotor for less than a full rotation.

7. The method of claim 5, wherein the predetermined period of time for pausing operation of the pumping device comprises a sufficient amount of time to allow the liquid in the pump set to fill a segment of the pump set behind the rotor.

8. The method of claim 1, further comprising associating a delivery routine stored in memory of the flow control apparatus with the recognized pump set.

9. The method of claim 1, wherein the liquid comprises a nutritional liquid.

10. The method of claim 9, wherein the nutritional liquid comprises a solution of blended food.

11. The method of claim 1, wherein the liquid has a rating in the inclusive range of 2-4 according to the IDDSI Framework.

12. A flow control apparatus for use with a pump set to deliver liquid from a liquid container through the pump set to a subject, the flow control apparatus comprising:
a pumping device capable of acting on the pump set to produce a liquid flow in the pump set during a feeding cycle; and
a controller in communication with the pumping device for controlling operation of the pumping device during the feeding cycle for producing the flow of the liquid in the pump set, the controller including a processor and a memory, the controller configured to execute in the processor a feeding routine to deliver a single aliquot of the liquid from the liquid container to the subject and pause operation of the pumping device for a predetermined period of time before delivering another single aliquot of liquid from the liquid container to the subject.

13. The flow control apparatus of claim 12, wherein the pumping device comprises a rotor including a plurality of rollers configured to contact the pump set to produce the single aliquots of liquid, and wherein the single aliquot is less than the volume of liquid that would be delivered by one full rotation of the rotor.

14. The flow control apparatus of claim 13 wherein the single aliquot comprises the volume of liquid disposed between adjacent rollers of the rotor.

15. The flow control apparatus of claim 12, wherein the controller is programmed to execute the feeding routine to deliver an entire prescribed volume of the liquid from the liquid container within the feeding cycle.

16. The flow control apparatus of claim 12, wherein the controller is programmed to execute the feeding routine upon recognizing the pump set is loaded on the flow control apparatus.

17. The flow control apparatus of claim 16, further comprising a reader operatively connected to the processor and configured to read the pump set to identify the feeding routine for delivering the liquid in the liquid container.

18. The flow control apparatus of claim 12 wherein the controller is configured to calculate the pause between delivery of successive aliquots based on an input of the volume of liquid to be delivered to the subject.

19. The flow control apparatus of claim 12 wherein the controller is configured to execute in the processor another feeding routine in which the pump device delivers multiple aliquots of liquid to the subject during a continuous operation of the pumping device.

20. A method of operating a flow control apparatus to deliver liquid to a subject using a pumping device of the flow control apparatus, the method comprising:
recognizing a pump set including a liquid container with a volume of liquid mounted to the flow control apparatus whereby the pump set is positioned to be acted on by the pumping device to deliver aliquots of liquid through the pump set;
initiating operation of the pumping device to draw a prescribed volume of the liquid from the liquid container for a duration of time; and
delivering a series of aliquots of the volume of liquid from the liquid container to the subject, wherein the series of aliquots is alternated with a series of pauses in operation of the pumping device for a predetermined period of time such that each aliquot in the series of aliquots is separated by a single pause in operation of the pumping device.

21. The method of claim 20, wherein the series of aliquots and the series of pauses in operation are at uniform intervals within the duration of time during which the prescribed volume of the liquid is pumped to the subject.

22. The method of claim 20, wherein the series of aliquots is alternated with a series of pauses in operation of the pumping device for a predetermined period of time without any additional pauses between aliquots of the volume of liquid in the duration of time while the prescribed volume of liquid is pumped to the subject.
